SEQUENCE LISTING (28 sequences)

SEQ ID NO: 1 - DNA, 128 bp, Artificial Sequence
  G719S-WT
gcttgtggagcctcttacacccagtggagaagctcccaaccaagctctcttgaggatcttgaaggaaactgaattcaaaaagatcaaagtgctgggctccggtgcgttcggcacggtgtataaggtaa

SEQ ID NO: 2 - DNA, 128 bp, Artificial Sequence
  G719S-MT
gcttgtggagcctcttacacccagtggagaagctcccaaccaagctctcttgaggatcttgaaggaaactgaattcaaaaagatcaaagtgctgagctccggtgcgttcggcacggtgtataaggtaa

SEQ ID NO: 3 - DNA, 18 bp, Artificial Sequence
  G719S-F
gcttgtggagcctcttac

SEQ ID NO: 4 - DNA, 19 bp, Artificial Sequence
  G719S-R
ttaccttatacaccgtgcc

SEQ ID NO: 5 - DNA, 16 bp, Artificial Sequence
  G719-WT-P
tgctgggctccggtgc

SEQ ID NO: 6 - DNA, 16 bp, Artificial Sequence
  G719-MT-P
tgctgagctccggtgc

SEQ ID NO: 7 - DNA, 117 bp, Artificial Sequence
  19del-WT
tgtcatagggactctggatcccagaaggtgagaaagttaaaattcccgtcgctatcaaggaattaagagaagcaacatctccgaaagccaacaaggaaatcctcgatgtgagtttct

SEQ ID NO: 8 - DNA, 102 bp, Artificial Sequence
  19del-MT
tgtcatagggactctggatcccagaaggtgagaaagttaaaattcccgtcgctatcaaaacatctccgaaagccaacaaggaaatcctcgatgtgagtttct

SEQ ID NO: 9 - DNA, 19 bp, Artificial Sequence
  19del-F1
tgtcatagggactctggat

SEQ ID NO: 10 - DNA, 20 bp, Artificial Sequence
  19del-R1
agaaactcacatcgaggatt

SEQ ID NO: 11 - DNA, 21 bp, Artificial Sequence
  19del-WT-P
gttgcttctcttaattccttg

SEQ ID NO: 12 - DNA, 19 bp, Artificial Sequence
  19del-MT-P
ggagatgttttgatagcga

SEQ ID NO: 13 - DNA, 122 bp, Artificial Sequence
  T790M-WT
ggaagcctacgtgatggccagcgtggacaacccccacgtgtgccgcctgctgggcatctgcctcacctccaccgtgcagctcatcacgcagctcatgcccttcggctgcctcctggactatg

SEQ ID NO: 14 - DNA, 122 bp, Artificial Sequence
  T790M-MT
ggaagcctacgtgatggccagcgtggacaacccccacgtgtgccgcctgctgggcatctgcctcacctccaccgtgcagctcatcatgcagctcatgcccttcggctgcctcctggactatg

SEQ ID NO: 15 - DNA, 17 bp, Artificial Sequence
  T790M-F
ggaagcctacgtgatgg

SEQ ID NO: 16 - DNA, 17 bp, Artificial Sequence
  T790M-R
catagtccaggaggcag

SEQ ID NO: 17 - DNA, 17 bp, Artificial Sequence
  T790M-WT-P
atgagctgcgtgatgag

SEQ ID NO: 18 - DNA, 17 bp, Artificial Sequence
  T790M-MT-P
atgagctgcatgatgag

SEQ ID NO: 19 - DNA, 128 bp, Artificial Sequence
  L858R-WT
ctacttggaggaccgtcgcttggtgcaccgcgacctggcagccaggaacgtactggtgaaaacaccgcagcatgtcaagatcacagattttgggctggccaaactgctgggtgcggaagagaaagaat

SEQ ID NO: 20 - DNA, 128 bp, Artificial Sequence
  L858R-MT
ctacttggaggaccgtcgcttggtgcaccgcgacctggcagccaggaacgtactggtgaaaacaccgcagcatgtcaagatcacagattttgggcgggccaaactgctgggtgcggaagagaaagaat

SEQ ID NO: 21 - DNA, 19 bp, Artificial Sequence
  L858R-F1
attctttctcttccgcacc

SEQ ID NO: 22 - DNA, 18 bp, Artificial Sequence
  L858R-R1
ctacttggaggaccgtcg

SEQ ID NO: 23 - DNA, 16 bp, Artificial Sequence
  L858R-WT-P
agtttggccagcccaa

SEQ ID NO: 24 - DNA, 16 bp, Artificial Sequence
  L858R-MT-P
agtttggcccgcccaa

SEQ ID NO: 25 - DNA, 13 bp, Artificial Sequence
  G719S WT-P1
cggagcccagcac

SEQ ID NO: 26 - DNA, 14 bp, Artificial Sequence
  G719S MT-P1
ccggagctcagcac

SEQ ID NO: 27 - DNA, 13 bp, Artificial Sequence
  G719S WT-P2
agtgctgggctcc

SEQ ID NO: 28 - DNA, 14 bp, Artificial Sequence
  G719S MT-P2
agtgctgagctccg

(19) United States
(12) Patent Application Publication
(10) Pub. No.: US 2025/0223648 A1
(43) Pub. Date: Jul. 10, 2025

(54) A primer-probe composition and a kit for detecting EGFR gene mutations

(71) Applicant: BOE TECHNOLOGY GROUP CO., LTD., Beijing (CN)

(72) Inventors: Kang PENG, Beijing (CN); Jiayu WANG, Beijing (CN); Meiping ZHAO, Beijing (CN); Shinying LAU, Beijing (CN); Wei CHEN, Beijing (CN); Zhukai LIU, Beijing (CN); Ding DING, Beijing (CN)

(73) Assignee: BOE TECHNOLOGY GROUP CO., LTD., Beijing (CN)

(21) Appl. No.: 18/283,896

(22) PCT Filed: Jun. 29, 2022
(86) PCT No.: PCT/CN2022/102367

§ 371 Date: Sep. 25, 2023

(51) Int. Cl. C12Q 1/6886; C12Q 1/6851

(52) U.S. Cl. CPC C12Q 1/6886; C12Q 1/6851; C12Q 2600/156

(57) ABSTRACT

The present disclosure provides a primer-probe composition for detecting an epidermal growth factor receptor (EGFR) gene mutation, a kit, a system and a device including the primer-probe composition, and the use thereof.

### CROSS REFERENCE TO RELATED APPLICATIONS

This application is the National Stage of PCT/CN2022/102367 filed on Jun. 29, 2022, the disclosure of which is incorporated by reference.

### REFERENCE TO AN ELECTRONIC SEQUENCE LISTING

The contents of the electronic sequence listing (sequencelisting.txt; Size: 6,420 bytes; and Date of Creation: Jan. 15, 2025) is herein incorporated by reference in its entirety.

### FIELD OF INVENTION

The present disclosure relates to the field of biotechnology and medical devices, in particular to a probe, a detection method, a kit, a system and a device for detecting an EGFR gene mutation, and the use of the above probe, detection method, kit, system and device to detect an EGFR gene mutation or to diagnose related diseases.

### BACKGROUND ART

EGFR (Epidermal Growth h Factor Receptor) is a transmembrane glycoprotein and one of the four members of the epidermal growth factor receptor family. EGFR has tyrosine kinase activity and is monomeric in the inactive state. When the EGFR binds to the ligand, EGFR dimerizes, and autophosphorylation of the intracytoplasmic EGFR tyrosine kinase domains occurs. The phosphorylated tyrosine kinases bind to the intracellular signaling proteins resulting in the activation of related signaling proteins, the promotion of cell growth, proliferation and differentiation (Merbst R. Int J Radiation Oncology Biol Phys, 2004, 59, 21).

The occurrence of various cancers is closely related to EGFR gene mutations. There are four main types of EGFR gene mutations, namely deletion mutation at exon 19, point mutation at exon 21, point mutation at exon 18, and insertion mutation at exon 20. Among them, the most common EGFR mutations are deletion mutation at exon 19, and L858R mutation at exon 21, which two mutations can lead to the activation of EGFR protein, promoting the proliferation and migration of tumor cells, and inhibiting the death of tumor cells.

Lung cancer is one of the types of cancers with the highest incidence in the world, of which 75%-80% is non-small cell lung cancer. Among the patients with non-small cell lung cancer in Asia, the incidence of EGFR mutations exceeds 40%, of which deletion at exon 19 and L858R mutation at exon 21 account for 80-90% of all the EGFR mutations. In addition, about 50% of the EGFR-positive patients have T790M mutation at exon 20 after administration of targeted drugs, which leads to the failure of targeted drugs.

At present, there are mainly the following methods for detecting gene mutations: (1) Direct sequencing by Sanger sequencing method (Sanger F. et al. Proc Natl Acad Sci, 1977, 74, 5463). This method is the gold standard for gene mutation detection, but its detection sensitivity is low, and only when the mutation abundance reaches more than 5% can it accurately detect mutations, which cannot meet the needs of liquid biopsy or early screening; (2) High-Resolution Melting Curve method (Montgometry J. et al. Nat Protoc, 2008, 14, 579). This method detects gene mutations through different melting curves formed by the combination of saturated DNA fluorescent dyes and PCR amplification products, and the detection sensitivity is about 1%; (3) Next-Generation Sequencing (NGS). This method can carry out large-scale screening of mutations, and its sensitivity can reach 0.1-0.5%, but the detection cost is high and the detection time is long, which limit its application for mutation detection in the clinical settings; (4) Amplification Refractory Mutation System (ARMS). Based on traditional PCR, this system uses specific primers to specifically amplify mutant DNA, and the detection sensitivity for mutant genes is about 1%; (5) Digital PCR (dPCR). Based on the TaqMan probe technology, this method divides the PCR reaction system into tens of thousands to hundreds of thousands of independent amplification systems with an array microwell plate or a droplet generator, which enables the absolute quantification of the mutant DNA copy number (Vogelstein B. et al. Proc Natl Acad Sci, 1999, 96, 9236). Its actual detection limit is between 0.1-0.5%, and compared with the NGS method, the detection time is short, the cost is low, and it is easier to be utilized widely in labs, hospitals, etc.

### SUMMARY OF THE INVENTION

The present disclosure provides a primer-probe composition for detecting epidermal growth factor receptor (EGFR) gene mutations, a kit, a system and a device comprising the primer-probe composition, and their uses in detection of EGFR mutations and diagnosis and treatment of diseases.

Accordingly, in one aspect, the present disclosure provides a primer-probe composition for detecting an epidermal growth factor receptor (EGFR) gene mutation, comprising a combination of primers and probes for one or more of the following gene mutations: G719S point mutation at exon 18, E746_A750del deletion mutation at exon 19, T790M point mutation at exon 20, and L858R point mutation at exon 21.

In some embodiments of the primer-probe composition of the present disclosure,
for G719S point mutation at exon 18, the primers are
G719S-F: GCTTGTGGAGCCTCTTAC (SEQ ID NO: 3), or
a nucleotide sequence in which one or more (for example 1-5, such as 1, 2, 3, 4 or 5) nucleotides are substituted, deleted, added and inserted in the nucleotide sequence shown in SEQ ID NO: 3, and
G719S-R: TTACCTTATACACCGTGCC (SEQ ID NO: 4), or
a nucleotide sequence in which one or more (for example 1-5, such as 1, 2, 3, 4 or 5) nucleotides are substituted, deleted, added and inserted in the nucleotide sequence shown in SEQ ID NO: 4;
the probes are
G719-WT-P: TGCTGGGCTCCGGTGC (SEQ ID NO: 5), or
a nucleotide sequence in which one or more (for example 1-5, such as 1, 2, 3, 4 or 5) nucleotides are substituted, deleted, added and inserted in the nucleotide sequence shown in SEQ ID NO: 5,
wherein a first fluorescent group is conjugated to the 5′ end of the G719-WT-P probe, and a minor groove binder is conjugated to the 3′ end of the G719-WT-P probe,
and
G719-MT-P: TGCTGAGCTCCGGTGC (SEQ ID NO: 6), or
a nucleotide sequence in which one or more (for example 1-5, such as 1, 2, 3, 4 or 5) nucleotides are substituted, deleted, added and inserted in the nucleotide sequence shown in SEQ ID NO: 6,
wherein a second fluorescent group is conjugated to the 5′ end of the G719-MT-P probe, and a minor groove binder is conjugated to the 3′ end of the G719-MT-P probe;
for E746_A750del deletion mutation at exon 19, the primers are
19del-F1: TGTCATAGGGACTCTGGAT (SEQ ID NO: 9), or
a nucleotide sequence in which one or more (for example 1-5, such as 1, 2, 3, 4 or 5) nucleotides are substituted, deleted, added and inserted in the nucleotide sequence shown in SEQ ID NO: 9, and
19del-R1: AGAAACTCACATCGAGGATT (SEQ ID NO: 10), or
a nucleotide sequence in which one or more (for example 1-5, such as 1, 2, 3, 4 or 5) nucleotides are substituted, deleted, added and inserted in the nucleotide sequence shown in SEQ ID NO: 10;
the probes are
19del-WT-P: GTTGCTTCTCTTAATTCCTTG (SEQ ID NO: 11), or
a nucleotide sequence in which one or more (for example 1-5, such as 1, 2, 3, 4 or 5) nucleotides are substituted, deleted, added and inserted in the nucleotide sequence shown in SEQ ID NO: 11,
wherein a first fluorescent group is conjugated to the 5′ end of the 19del-WT-P probe, and a minor groove binder is conjugated to the 3′ end of the 19del-WT-P probe, and
19del-MT-P: GGAGATGTTTTGATAGCGA (SEQ ID NO: 12), or
a nucleotide sequence in which one or more (for example 1-5, such as 1, 2, 3, 4 or 5) nucleotides are substituted, deleted, added and inserted in the nucleotide sequence shown in SEQ ID NO: 12,
wherein a second fluorescent group is conjugated to the 5′ end of the 19del-MT-P probe, and a minor groove binder is conjugated to the 3′ end of the 19del-MT-P probe;
for T790M point mutation at exon 20, the primers are
T790M-F: GGAAGCCTACGTGATGG (SEQ ID NO: 15), or
a nucleotide sequence in which one or more (for example 1-5, such as 1, 2, 3, 4 or 5) nucleotides are substituted, deleted, added and inserted in the nucleotide sequence shown in SEQ ID NO: 15, and
T790M-R: CATAGTCCAGGAGGCAG (SEQ ID NO: 16), or
a nucleotide sequence in which one or more (for example 1-5, such as 1, 2, 3, 4 or 5) nucleotides are substituted, deleted, added and inserted in the nucleotide sequence shown in SEQ ID NO: 16;
the probes are
T790M-WT-P: ATGAGCTGCGTGATGAG (SEQ ID NO: 17), or
a nucleotide sequence in which one or more (for example 1-5, such as 1, 2, 3, 4 or 5) nucleotides are substituted, deleted, added and inserted in the nucleotide sequence shown in SEQ ID NO: 17,
wherein a first fluorescent group is conjugated to the 5′ end of the T790M-WT-P probe, and a minor groove binder is conjugated to the 3′ end of the T790M-WT-P probe, and
T790M-MT-P: ATGAGCTGCATGATGAG (SEQ ID NO: 18), or
a nucleotide sequence in which one or more (for example 1-5, such as 1, 2, 3, 4 or 5) nucleotides are substituted, deleted, added and inserted in the nucleotide sequence shown in SEQ ID NO: 18,
wherein a second fluorescent group is conjugated to the 5′ end of the T790M-WT-P probe, and a minor groove binder is conjugated to the 3′ end of the T790M-WT-P probe;
for L858R point mutation at exon 21, the primers are
L858R-F1: ATTCTTTCTCTTCCGCACC (SEQ ID NO: 21), or
a nucleotide sequence in which one or more (for example 1-5, such as 1, 2, 3, 4 or 5) nucleotides are substituted, deleted, added and inserted in the nucleotide sequence shown in SEQ ID NO: 21, and
L858R-R1: CTACTTGGAGGACCGTCG (SEQ ID NO: 22), or
a nucleotide sequence in which one or more (for example 1-5, such as 1, 2, 3, 4 or 5) nucleotides are substituted, deleted, added and inserted in the nucleotide sequence shown in SEQ ID NO: 22;
the probes are
L858R-WT-P: AGTTTGGCCAGCCCAA (SEQ ID NO: 23), or
a nucleotide sequence in which one or more (for example 1-5, such as 1, 2, 3, 4 or 5) nucleotides are substituted, deleted, added and inserted in the nucleotide sequence shown in SEQ ID NO: 23,
wherein a first fluorescent group is conjugated to the 5′ end of the L858R-WT-P probe, and a minor groove binder is conjugated to the 3′ end of the L858R-WT-P probe, and
L858R-MT-P: AGTTTGGCCCGCCCAA (SEQ ID NO: 24), or
a nucleotide sequence in which one or more (for example 1-5, such as 1, 2, 3, 4 or 5) nucleotides are substituted, deleted, added and inserted in the nucleotide sequence shown in SEQ ID NO: 24,
wherein a second fluorescent group is conjugated to the 5′ end of the L858R-WT-P probe, and a minor groove binder is conjugated to the 3′ end of the L858R-WT-P probe;
wherein the first fluorescent group and the second fluorescent group are respectively selected from Alexa Fluor 488, FAM, TET, JOE, VIC and HEX, and the first fluorescent group and the second fluorescent group are different.

In another aspect, the present disclosure provides a kit for detecting an EGFR gene mutation, comprising the primer-probe composition of the present disclosure, wherein the gene mutation is selected from one or more of G719S point mutation at exon 18, E746_A750del deletion mutation at exon 19, T790M point mutation at exon 20, and L858R point mutation at exon 21.

In some embodiments, the kit of the present disclosure further comprises a PCR master mix and a dNTP mix. In some embodiments, the final concentration of each primer in the kit of the present disclosure is 450-900 nM, such as 450 nM, 500 nM, 550 nM, 600 nM, 650 nM, 700 nM, 750 nM, 800 nM, 850 nM or 900 nM. In some embodiments, the final concentration of each probe in the kit of the present disclosure is 500-700 nM, such as 500 nM, 550 nM, 600 nM, 650 nM or 700 nM. In some embodiments, the final concentration of the PCR master mix in the kit of the present disclosure is 1-1.5×, such as 1.2×. In some embodiments, the final concentration of the dNTP mix in the kit of the present disclosure is 0-125 μM, such as 5, 15, 30, 45, 60, 75, 90, 105, 120 or 125 μM.

In yet another aspect, the present disclosure provides a method for detecting an EGFR gene mutation in a DNA sample based on digital PCR, wherein the gene mutation is selected from one or more of G719S point mutation at exon 18, E746_A750del deletion mutation at exon 19, T790M point mutation at exon 20, and L858R point mutation at exon 21. In some embodiments, the method comprises:
i) providing the primer-probe composition of the present disclosure for the EGFR gene mutation to be detected,
ii) mixing the DNA sample, the primer-probe composition provided in i), a PCR master mix and a dNTP mix to obtain a detection solution,
iii) loading the detection solution on a chip for PCR amplification reaction, and
iv) collecting and processing signals from the products of the PCR amplification reaction on the chip to detect whether the EGFR gene mutation is present in the DNA sample.

In some embodiments of the method of the present disclosure, the procedure of the PCR amplification reaction in iii) is
a) 92-98° C. for 2-8 minutes, for example 4-6 minutes, such as 5 minutes,
b) 92-98° C. for 15-40 seconds, for example 20-30 seconds, such as 25 seconds,
c) 52-58° C. for 20-50 seconds, for example 30-40 seconds, such as 35 seconds,
d) 68-76° C. for 40-80 seconds, for example 50-70 seconds, such as 60 seconds, and
e) 2-10° C. to terminate the reaction,
wherein the steps b)-d) are performed for 30-50 cycles, for example 35-45 cycles, such as 42 cycles.

In some embodiments of the method of the present disclosure, in the detection solution,
the DNA sample is at the final concentration of 300-30000 copies/μL, for example 300, 3000 or 30000 copies/μL,
both forward and reverse primers of the primers are at the final concentrations of 300-600 nM, for example 400-500 nM, such as 450 nM, and
the probe is at the final concentration of 400-600 nM, such as 500 nM.

In some embodiments of the method of the present disclosure, the mutation abundance in the DNA template is 0.5% or more.

In another aspect, the present disclosure provides a system for detecting an EGFR gene mutation, comprising the primer-probe composition of the present disclosure, wherein the gene mutation is selected from one or more of G719S point mutation at exon 18, E746_A750del deletion mutation at exon 19, T790M point mutation at exon 20, and L858R point mutation at exon 21. In some preferred embodiments, the system is an array or chip, wherein the primer-probe composition of the present disclosure is immobilized on the array or chip.

In another aspect, the present disclosure provides a device for detecting an EGFR gene mutation in a DNA sample, comprising the system of the present disclosure. In some preferred embodiments, the device is a chip-based digital PCR machine.

In yet another aspect, the present disclosure provides the uses of the primer-probe composition, the kit, the system or the device of the present disclosure for detecting an EGFR gene mutation in a DNA sample, wherein the gene mutation is selected from one or more of G719S point mutation at exon 18, E746_A750del deletion mutation at exon 19, T790M point mutation at exon 20, and L858R point mutation at exon 21.

In yet another aspect, the present disclosure provides the uses of the primer-probe composition, the kit, the system, or the device of the present disclosure for diagnosing diseases.

In another aspect, the present disclosure provides the primer-probe composition, the kit, the system, or the device of the present disclosure for use in diagnosing diseases.

In another aspect, the present disclosure provides the use of the primer-probe composition of the present disclosure in the manufacture of a composition for diagnosing diseases.

In some embodiments of the above aspects, the diseases are cancers, e.g., lung cancer (such as lung adenocarcinoma, non-small cell lung cancer, or small cell lung cancer), hematologic malignancy, pancreatic cancer, colorectal cancer, or malignant glioma.

The primer-probe composition, the kit, the system or the device provided in the present disclosure, as well as the detection method using the same, can specifically detect four EGFR mutation sites. Compared with other mutation detection methods in the prior art, the present invention has the advantages such as, short detection time, simple operation, low detection cost and high sensitivity. The technology of the present disclosure can accurately and effectively detect mutant genes with a mutation abundance of 0.5%, and its sensitivity is significantly higher than that of Sanger sequencing, High-Resolution Melting Curve method, etc., and is comparable to the sensitivity of NGS and digital droplet PCR (ddPCR) method. Taking the detection time or the difficulty of operation into consideration, the time of the detection of the present disclosure is short and the operation is simple. Taking the cost into consideration, the detection cost of the present disclosure is lower than that of NGS and the digital droplet PCR method.

### DESCRIPTION OF THE DRAWINGS

The characteristics and advantages of the invention may be understood by reference to the following detailed description of the exemplary embodiments utilizing the principles of the invention and to the accompanying drawings, in which:

FIG. 1 is schematic diagrams of the results of ddPCR for detecting the G719S mutation using the first, second and third versions of the probes, in which (a) shows the detection result of the first version of the mutant probe; (b) shows the detection result of the first version of the wild-type probe; (c) shows the detection result of the second version of the mutant probe; (d) shows the detection result of the second version of the wild-type probe; (e) shows the detection result of the third version of the mutant probe; and (f) shows the detection result of the third version of the wild-type probe.

FIG. 2 is schematic diagrams of the results of detecting the standard for deletion mutation at exon 19 with a mutation abundance of 10% based on digital PCR chip, in which (a) shows the fluorescent microscope photo before amplification; (b) shows the fluorescent microscope photo after amplification; and (c) shows the result obtained after data processing on the chip area after amplification.

FIG. 3 is schematic diagrams of the results of detecting the standard for deletion mutation at exon 19 with a mutation abundance of 0.5% based on digital PCR chip, in which (a) shows the fluorescent microscope photo before amplification; (b) shows the fluorescent microscope photo after amplification; and (c) shows the result obtained after data processing on the chip area after amplification.

FIG. 4 is schematic diagrams of the results of detecting the standard for T790M point mutation at exon 20 with a mutation abundance of 10% based on digital PCR chip, in which (a) shows the fluorescent microscope photo before amplification; (b) shows the fluorescent microscope photo after amplification; and (c) shows the result obtained after data processing on the chip area after amplification.

FIG. 5 is schematic diagrams of the results of detecting the standard for T790M point mutation at exon 20 with a mutation abundance of 0.5% based on digital PCR chip, in which (a) shows the fluorescent microscope photo before amplification; (b) shows the fluorescent microscope photo after amplification; and (c) shows the result obtained after data processing on the chip area after amplification.

FIG. 6 is schematic diagrams of the results of detecting the standard for G719S point mutation at exon 18 with a mutation abundance of 10% based on digital PCR chip, in which (a) shows the fluorescent microscope photo before amplification; (b) shows the fluorescent microscope photo after amplification; and (c) shows the result obtained after data processing on the chip area after amplification.

FIG. 7 is schematic diagrams of the results of detecting the standard for G719S point mutation at exon 18 with a mutation abundance of 0.5% based on digital PCR chip, in which (a) shows the fluorescent microscope photo before amplification; (b) shows the fluorescent microscope photo after amplification; and (c) shows the result obtained after data processing on the chip area after amplification.

FIG. 8 is schematic diagrams of the results of detecting the standard for L858R point mutation at exon 21 with a mutation abundance of 10% based on digital PCR chip, in which (a) shows the fluorescent microscope photo before amplification; (b) shows the fluorescent microscope photo after amplification; and (c) shows the result obtained after data processing on the chip area after amplification.

FIG. 9 is schematic diagrams of the results of detecting the standard for L858R point mutation at exon 21 with a mutation abundance of 0.5% based on digital PCR chip, in which (a) shows the fluorescent microscope photo before amplification; (b) shows the fluorescent microscope photo after amplification; and (c) shows the result obtained after data processing on the chip area after amplification.

FIG. 10 is schematic diagrams of the results of detecting the purified nucleic acids extracted from the formaldehyde-fixed paraffin-embedded (FFPE) samples containing 19del deletion mutation based on digital PCR chip, in which (a) shows the fluorescent microscope photo before amplification; (b) shows the fluorescent microscope photo after amplification; and (c) shows the result obtained after data processing on the chip area after amplification.

### DETAILED EMBODIMENTS

The present invention is further illustrated by the following examples, but any example or the combination thereof should not be construed as a limit to the scope or embodiments of the present invention. The scope of the present invention is defined by the appended claims, and those skilled in the art can clearly understand the scope defined by the claims in combination with the description of the invention and common knowledge in the field. Without departing from the spirit and scope of the present invention, those skilled in the art may make any modifications or changes to the technical solutions of the present invention, and such modifications and changes are also included in the scope of the present invention.

### Example 1. Design and Preparation of Primer Pairs and Probes

The present disclosure designs primers and probes based on the following four mutation sites for EGFR mutant genes: deletion mutation at exon 19, L858R point mutation at exon 21, T790M point mutation at exon 20, and G719S point mutation at exon 18. Details of the mutations are shown in Table 1 below.

TABLE 1
Mutation sites corresponding to the
detection of EGFR gene mutations

| | Amino Acid | | | |
|---|---|---|---|---|
| Mutation Name | Change | Exon | Base Change | Cosmic ID |
| Exon 18 G719S | G719S | 18 | c.2155G > A | 6252 |
| Exon 19 del | E746_A750del | 19 | c.2235_2249 | 6223 |
| | | | del 15 | |
| Exon 20 T790M | T790M | 20 | c.2369C > T | 6240 |
| Exon 21 L858R | L858R | 21 | c.2573T > G | 6224 |

Wild-type (WT) and mutant (MT) DNA sequences containing the four mutation sites for EGFR mutant genes in Table 1 are shown in Table 2 below, in which the mutation sites are underlined in bold.

TABLE 2
Wild-type and mutant DNA sequences containing
mutation sites for EGFR gene

| Detection | | | SEQ ID |
|---|---|---|---|
| Site | Name | Sequence (5′-3′) | NO |
| Exon 18 | G719S-WT | GCTTGTGGAGCCTCTTACACCCAGTGGAGAAG | 1 |
| G719S | | CTCCCAACCAAGCTCTCTTGAGGATCTTGAAG | |
| | | GAAACTGAATTCAAAAAGATCAAAGTGCTGGG | |
| | | CTCCGGTGCGTTCGGCACGGTGTATAAGGTAA | |
| | G719S-MT | GCTTGTGGAGCCTCTTACACCCAGTGGAGAAG | 2 |
| | | CTCCCAACCAAGCTCTCTTGAGGATCTTGAAG | |
| | | GAAACTGAATTCAAAAAGATCAAAGTGCTGAG | |
| | | CTCCGGTGCGTTCGGCACGGTGTATAAGGTAA | |
| Exon 19 | 19del-WT | TGTCATAGGGACTCTGGATCCCAGAAGGTGAG | 7 |
| del | | AAAGTTAAAATTCCCGTCGCTATCAAGGAATT | |
| | | AAGAGAAGCAACATCTCCGAAAGCCAACAAG | |
| | | GAAATCCTCGATGTGAGTTTCT | |
| | 19del-MT | TGTCATAGGGACTCTGGATCCCAGAAGGTGAG | 8 |
| | | AAAGTTAAAATTCCCGTCGCTATCAAAACATCT | |
| | | CCGAAAGCCAACAAGGAAATCCTCGATGTGAG | |
| | | TTTCT | |
| Exon 20 | T790M-WT | GGAAGCCTACGTGATGGCCAGCGTGGACAACC | 13 |
| T790M | | CCCACGTGTGCCGCCTGCTGGGCATCTGCCTC | |
| | | ACCTCCACCGTGCAGCTCATCACGCAGCTCAT | |
| | | GCCCTTCGGCTGCCTCCTGGACTATG | |
| | T790M-MT | GGAAGCCTACGTGATGGCCAGCGTGGACAACC | 14 |
| | | CCCACGTGTGCCGCCTGCTGGGCATCTGCCTC | |
| | | ACCTCCACCGTGCAGCTCATCATGCAGCTCAT | |
| | | GCCCTTCGGCTGCCTCCTGGACTATG | |
| Exon 21 | L858R-WT | CTACTTGGAGGACCGTCGCTTGGTGCACCGCG | 19 |
| L858R | | ACCTGGCAGCCAGGAACGTACTGGTGAAAAC | |
| | | ACCGCAGCATGTCAAGATCACAGATTTTGGGC | |
| | | TGGCCAAACTGCTGGGTGCGGAAGAGAAAGA | |
| | | AT | |
| | L858R-MT | CTACTTGGAGGACCGTCGCTTGGTGCACCGCG | 20 |
| | | ACCTGGCAGCCAGGAACGTACTGGTGAAAAC | |
| | | ACCGCAGCATGTCAAGATCACAGATTTTGGGC | |
| | | GGGCCAAACTGCTGGGTGCGGAAGAGAAAGA | |
| | | AT | |

In the following, the detection of G719S mutation at exon 18 is taken as an example to illustrate the screening process of probes.

Three versions of probes were designed for G719S mutation at exon 18 using a conventional probe design software, and the sequences are shown in Table 3 below.

TABLE 3
Detection probes for mutation sites of
G719S mutation at exon 18

| Version | | | Tm | SEQ |
|---|---|---|---|---|
| No. | Name | Sequence (5′-3′) | Value | ID NO |
| First | G719S WT-P1 | CGGAGCCCAGCAC | 58.2 | 25 |
| Version | G719S MT-P1 | CCGGAGCTCAGCAC | 58.5 | 26 |
| Second | G719S WT-P2 | AGTGCTGGGCTCC | 56.4 | 27 |
| Version | G719S MT-P2 | AGTGCTGAGCTCCG | 57.3 | 28 |
| Third | G719S WT-P3 | TGCTGGGCTCCGGTGC | 67.9 | 5 |
| Version | G719S MT-P3 | TGCTGAGCTCCGGTGC | 64.8 | 6 |

According to the manufacturer's instruction, the detection results of each version of the probes were compared using the digital droplet PCR (ddPCR) method, and the detection platform was Bio-rad ddPCR platform (Bio-rad QX200 ddPCR instrument), the detection objects were the wild-type and mutant DNA double-stranded standard templates containing the G719S site (sequences are shown in Table 2).

Specifically, the experimental procedure is outlined as follows:
1. Preparation of a reaction solution: preparing a 20 uL reaction solution with the primers, probes, templates, and ddPCR master mix, and adding it to a droplet generation card matched with the instrument;
2. Preparation of droplets: placing the droplet generation card into a droplet generator, dividing each 20 uL reaction solution into 20,000 droplets in 2.5 mins;
3. PCR amplification: transferring the droplets to a 96-well PCR plate, and performing amplification in a PCR machine, with the reaction conditions the same as other experiments;
4. Droplet detection: transferring the plate to a droplet analyzer, sequentially taking and passing the droplets of each sample one by one through the detector; and
5. Analyzing data: among the droplets passing through the detector, the droplets with fluorescent signals are positive, and the droplets without fluorescent signals are negative, and the software records the proportion of positive droplets in each sample; then analyzing the data and displaying the detection results.

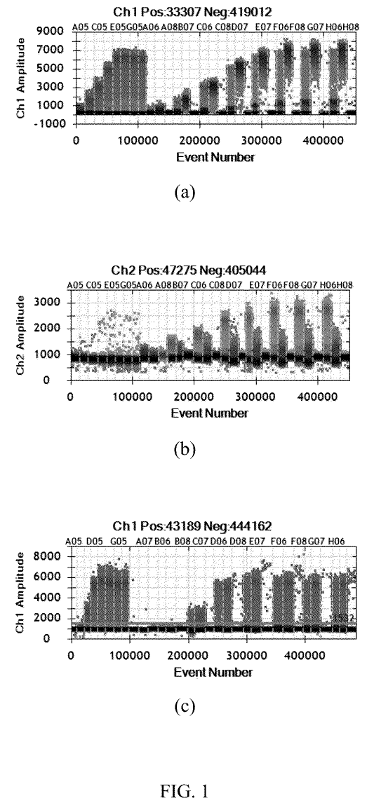

The detection results of the three versions of the probes are shown in FIG. 1.

The results indicate: the detection results of the first version of the probes ((a) and (b) in FIG. 1) show that the signal of the wild-type strand is normal, but the signal of the mutant strand is less, indicating that the binding efficiency of the probes to the mutant templates is low; with the complementary strand of the templates serving as the binding objects of the probes for the second version of the probes, the detection results ((c) and (d) in FIG. 1) show that the signal of the mutant strand is normal, but there is no amplification signal for the wild-type strand, indicating that the binding efficiency of the probes to the wild-type template is low; the binding region is moved to the upstream of the template strand for the third version of the probes, the length of the probes is increased, and the Tm value and GC content are improved, contributing to the binding with the template strand. The detection results ((e) and (f) in FIG. 1) show that both the mutant strand and the wild-type strand have signals, and a mutation abundance of 0.1% can be detected.

Referring to articles and primer databases, a conventional primer design software was used to design and optimize primers. Based on the probe design and screening method shown above, the specific primer pairs and probes for detecting the mutation sites at exon 18, exon 19, exon 20 and exon 21 of the human epidermal growth factor receptor (EGFR) gene shown in Table 2 were finally obtained. The sequences of the specific primer pairs and probes are shown in Table 4 below.

TABLE 4
Sequences of specific primers and probes for
detecting EGFR gene mutations

| De- | | | SEQ |
|---|---|---|---|
| tection | | | ID |
| Site | Name | Sequence (5′-3′) | NO |
| Exon 18 | G719S-F | GCTTGTGGAGCCTCTTAC | 3 |
| G719S | G719S-R | TTACCTTATACACCGTGCC | 4 |
| | G719-WT-P | HEX-TGCTGGGCTCCGGTGC-MGB | 5 |
| | G719-MT-P | FAM-TGCTGAGCTCCGGTGC-MGB | 6 |
| Exon 19 | 19del-F1 | TGTCATAGGGACTCTGGAT | 9 |
| del | 19del-R1 | AGAAACTCACATCGAGGATT | 10 |
| | 19del-WT-P | HEX-GTTGCTTCTCTTAATTCCTTG-MGB | 11 |
| | 19del-MT-P | FAM-GGAGATGTTTTGATAGCGA-MGB | 12 |
| Exon 20 | T790M-F | GGAAGCCTACGTGATGG | 15 |
| T790M | T790M-R | CATAGTCCAGGAGGCAG | 16 |
| | T790M-WT-P | HEX-ATGAGCTGCGTGATGAG-MGB | 17 |
| | T790M-MT-P | FAM-ATGAGCTGCATGATGAG-MGB | 18 |
| Exon 21 | L858R-F1 | ATTCTTTCTCTTCCGCACC | 21 |
| L858R | L858R-R1 | CTACTTGGAGGACCGTCG | 22 |
| | L858R-WT-P | HEX-AGTTTGGCCAGCCCAA-MGB | 23 |
| | L858R-MT-P | FAM-AGTTTGGCCCGCCCAA-MGB | 24 |

As shown in Table 4, specific probes also include fluorophores (FAM, HEX) and minor groove binders (MGB). In the present disclosure, the probes specifically hybridize with the target detection fragments, and the 5′ exonuclease activity of Taq polymerase is used to hydrolyze the probes to emit fluorescence during the PCR extension process, thereby the mutant sequences are detected.

Standard DNA templates used in the following examples are double-stranded DNA samples synthesized according to the DNA sequences shown in Table 2. In practical applications, there is no special requirement on the origin of the DNA templates, and the DNA templates are preferably extracted from formaldehyde-fixed paraffin-embedded (FFPE) samples. The DNA extraction steps of the paraffin-embedded samples are carried out according to the instructions for the kit, and more preferably, the TIANGEN paraffin-embedded tissue DNA rapid extraction kit, TIANquick FFPE DNA kit, is used for extraction.

The chip-based digital PCR system used in the examples is in 20 μL, preferably comprising 12 μL of PCR master mix, 1.8-2 μL of each primer, 1.4-2 μL of probes, 2 μL of DNA template, and with the remaining of the volume supplemented with ddH₂O.

The procedure of fluorescent PCR amplification utilized preferably includes: pre-denaturation at 95° C. for 5 mins; denaturation at 95° C. for 25 s, annealing at 55° C. for 35 s, extension at 72° C. for 60 s, for 42 cycles. After PCR, the fluorescent signals (FAM or HEX) corresponding to the probes are collected for further result analysis.

### Example 2. Detection Method of T790M Mutation Standard Sample

The detailed implementation steps of detection based on digital PCR chip are as follows:

### 1. Sample Preparation

(1) Synthesizing the standard sample sequences shown in Table 2 according to a standard method;
(2) Taking standard samples of wild-type DNA and mutant DNA, diluting the wild-type DNA to 6×10⁵ copies/μL with ddH₂O containing 0.1% Tween-20, and diluting the mutant DNA to 6×10⁴ copies/μL or 6×10³ copies/μL; and
(3) Preparing the detection solution, in which the components of the solution are shown in Table 5-1 to Table 5-3, and the loading concentrations of the mutant templates are 0, 300, and 3,000 copies/μL respectively (the loading concentrations of the mutant templates can actually be adjusted according to the detection capability of the chip).

TABLE 5-1
List of components of the detection
solution for T790M control system

| | Stock Solution | | | | Final | |
|---|---|---|---|---|---|---|
| Component | Concentration | | Volume | | Concentration | |
| PCR master mix | 2X | | 12 | μL | 1.2X | |
| T790M-F | 9 | μM | 1 | μL | 450 | nM |
| T790M-R | 9 | μM | 1 | μL | 450 | nM |
| T790M-MT-P | 5 | μM | 2 | μL | 500 | nM |
| dNTPs | 2.5 | mM | 1 | μL | 125 | μM |

| | | | | |
|---|---|---|---|---|
| wild-type DNA | 6 × 10⁵ copies/μL | 1 | μL | 3 × 10⁴ copies/μL |

| | | | |
|---|---|---|---|
| mutant DNA | 6 × 10⁴ copies/μL | 0 | 0 |
| ddH₂O | / | 2 | / |
| total volume | / | 20 | / |

TABLE 5-2
List of components of the detection solution
for T790M 0.5% mutation system

| | Stock Solution | | | | Final | |
|---|---|---|---|---|---|---|
| Component | Concentration | | Volume | | Concentration | |
| PCR master mix | 2X | | 12 | μL | 1.2X | |
| T790M-F | 9 | μM | 1 | μL | 450 | nM |
| T790M-R | 9 | μM | 1 | μL | 450 | nM |
| T790M-MT-P | 5 | μM | 2 | μL | 500 | nM |
| dNTPs | 2.5 | mM | 1 | μL | 125 | μM |

| | | | | |
|---|---|---|---|---|
| wild-type DNA | 6 × 10⁵ copies/μL | 2 | μL | 6 × 10⁴ copies/μL |
| mutant DNA | 6 × 10³ copies/μL | 1 | μL | 3 × 10² copies/μL |

| | | | |
|---|---|---|---|
| ddH₂O | / | 0 | / |
| total volume | / | 20 | / |

TABLE 5-3
List of components of the detection solution
for T790M 10% mutation system

| | Stock Solution | | | | Final | |
|---|---|---|---|---|---|---|
| Component | Concentration | | Volume | | Concentration | |
| PCR master mix | 2X | | 12 | μL | 1.2X | |
| T790M-F | 9 | μM | 1 | μL | 450 | nM |
| T790M-R | 9 | μM | 1 | μL | 450 | nM |
| T790M-MT-P | 5 | μM | 2 | μL | 500 | nM |
| dNTPs | 2.5 | mM | 1 | μL | 125 | μM |

| | | | | |
|---|---|---|---|---|
| wild-type DNA | 6 × 10⁵ copies/μL | 1 | μL | 3 × 10⁴ copies/μL |
| mutant DNA | 6 × 10⁴ copies/μL | 1 | μL | 3 × 10³ copies/μL |

| | | | |
|---|---|---|---|
| ddH₂O | / | 0 | / |
| total volume | / | 20 | / |

### 2. Chip Loading and Digital PCR Amplification

After the preparation of the solution, the chip was loaded and mounted. The loading volume was about 6.5-7 μL; and the temperature control program was set as follows: pre-denaturation at 95° C. for 5 mins; denaturation at 95° C. for 25 s, annealing at 55° C. for 35 s, extension at 72° C. for 60 s, for 42 cycles.

### 3. Fluorescent Signal Reading and Data Processing

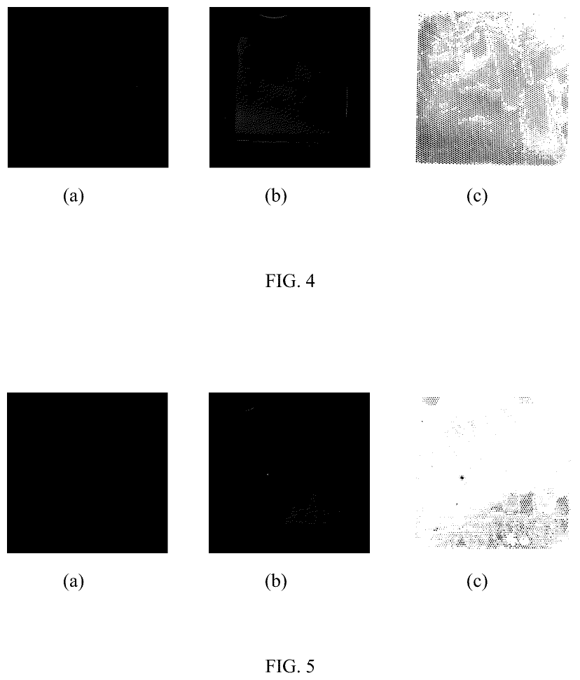

The chip was placed in the reader and fluorescent photos were taken, with the fluorescence channel set as FAM (excitation at about 488 nm, emission at about 525 nm), and the exposure time set as 2-5 s. The collected fluorescent dot matrix images were processed using ImageJ software, and the number of fluorescent bright spots (positive) was counted. Finally, the copy number concentration c of the sample was calculated according to the Poisson distribution formula c=−ln(1−p)/v, where p is the proportion of positive wells, and v is the volume of a single microwell. According to the results of the detection solution for the T790M 0.5% and 10% mutant systems, the measured concentrations of the mutant templates obtained from calculation are 363.8 copies/μL and 2,836 copies/μL, respectively, and the measured ratios of the mutant templates are 0.606% and 9.45%, respectively. Fluorescent signal results and data processing images are shown in FIG. 4 and FIG. 5.

The results indicate that it is effective to use the primers and probes for T790M mutation site of the present disclosure, and the reaction system can be used for the detection of samples containing this mutation site.

### Example 3. Detection Method of G719S Mutation Standard Sample

The detailed implementation steps of detection based on digital PCR chip are as follows:

### 1. Sample Preparation

(1) Synthesizing the standard sample sequences shown in Table 2 according to a standard method;
(2) Taking standard samples of wild-type DNA and mutant DNA, diluting the wild-type DNA to 6×10⁵ copies/μL with ddH₂O containing 0.1% Tween-20, and diluting the mutant DNA to 6×10⁴ copies/μL or 6×10³ copies/μL; and
(3) Preparing the detection solution, in which the components of the solution are shown in Table 5-1 to Table 5-3 (the sequences for primers, probes, and templates are replaced with the corresponding ones for G719S mutation), and the loading concentrations of the mutant templates are 0, 200, and 3,000 copies/μL respectively (the loading concentrations of the mutant templates can actually be adjusted according to the detection capability of the chip), and correspondingly, the loading concentrations of the wild-type templates are 30,000, 40,000, and 30,000 copies/μL respectively.

### 2. Chip Loading and Digital PCR Amplification

After the preparation of the solution, the chip was loaded and mounted. The loading volume was about 6.5-7 μL; and the temperature control program was set as follows: pre-denaturation at 95° C. for 5 mins; denaturation at 95° C. for 25 s, annealing at 55° C. for 35 s, extension at 72° C. for 60 s, for 42 cycles.

### 3. Fluorescent Signal Reading and Data Processing

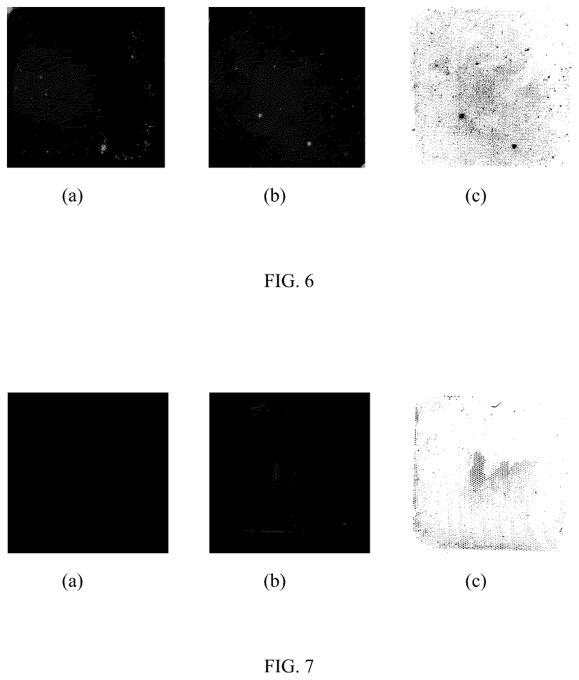

The chip was placed in the reader and fluorescent photos were taken, with the fluorescence channel set as FAM (excitation at about 488 nm, emission at about 525 nm), and the exposure time set as 2-5 s. The collected fluorescent dot matrix images were processed using ImageJ software, and the number of fluorescent bright spots (positive) was counted. Finally, the copy number concentration c of the sample was calculated according to the Poisson distribution formula c=−ln(1−p)/v, where p is the proportion of positive wells, and v is the volume of a single microwell. According to the results of the detection solution for the G719S 0.5% and 10% mutant systems, the measured concentrations of the mutant templates obtained from calculation are 281.2 copies/μL and 3,071 copies/μL, respectively, and the measured ratios of the mutant templates are 0.703% and 10.24%, respectively. Fluorescent signal results and data processing images are shown in FIG. 6 and FIG. 7.

The results indicate that it is effective to use the primers and probes for G719S mutation site of the present disclosure, and the reaction system can be used for the detection of samples containing this mutation site.

### Example 4. Detection Method of 19Del Mutation Standard Sample

The detailed implementation steps of detection based on digital PCR chip are as follows:

### 1. Sample Preparation

(1) Synthesizing the standard sample sequences shown in Table 2 according to a standard method;
(2) Taking standard samples of wild-type DNA and mutant DNA, diluting the wild-type DNA to 6×10⁵ copies/μL with ddH₂O containing 0.1% Tween-20, and diluting the mutant DNA to 6×10⁴ copies/μL or 6×10³ copies/μL; and
(3) Preparing the detection solution, in which the components of the solution are shown in Table 5-1 to Table 5-3 (the sequences for primers, probes, and templates are replaced with the corresponding ones for 19del mutation), and the loading concentrations of the mutant templates are 0, 300, and 3,000 copies/μL respectively (the loading concentrations of the mutant templates can actually be adjusted according to the detection capability of the chip).

### 2. Chip Loading and Digital PCR Amplification

After the preparation of the solution, the chip was loaded and mounted. The loading volume was about 6.5-7 μL; and the temperature control program was set as follows: pre-denaturation at 95° C. for 5 mins; denaturation at 95° C. for 25 s, annealing at 55° C. for 35 s, extension at 72° C. for 60 s, for 42 cycles.

### 3. Fluorescent Signal Reading and Data Processing

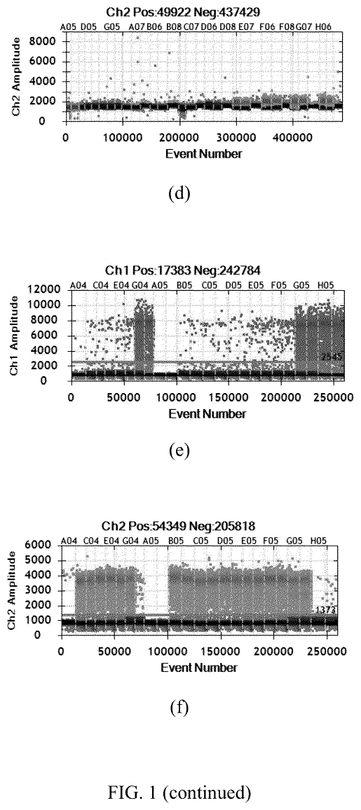

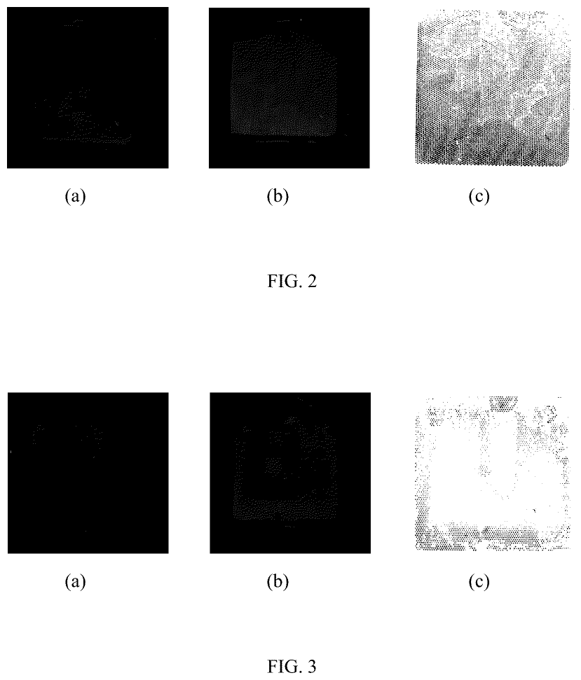

The chip was placed in the reader and fluorescent photos were taken, with the fluorescence channel set as FAM (excitation at about 488 nm, emission at about 525 nm), and the exposure time set as 2-5 s. The collected fluorescent dot matrix images were processed using ImageJ software, and the number of fluorescent bright spots (positive) was counted. Finally, the copy number concentration c of the sample was calculated according to the Poisson distribution formula c=−ln(1−p)/v, where p is the proportion of positive wells, and v is the volume of a single microwell. According to the results of the detection solution for the 19del 0.5% and 10% mutant systems, the measured concentrations of the mutant templates obtained from calculation are 387.7 copies/μL and 2,929 copies/μL, respectively, and the measured ratios of the mutant templates are 0.646% and 9.76%, respectively. Fluorescent signal results and data processing images are shown in FIG. 2 and FIG. 3.

The results indicate that it is effective to use the primers and probes for 19del mutation site of the present disclosure, and the reaction system can be used for the detection of samples containing this mutation site.

### Example 5. Detection Method of L858R Mutation Standard Sample

The detailed implementation steps of detection based on digital PCR chip are as follows:

### 1. Sample Preparation

(1) Synthesizing the standard sample sequences shown in Table 2 according to a standard method;
(2) Taking standard samples of wild-type DNA and mutant DNA, diluting the wild-type DNA to 6×10⁵ copies/μL with ddH₂O containing 0.1% Tween-20, and diluting the mutant DNA to 6×10⁴ copies/μL or 6×10³ copies/μL; and
(3) Preparing the detection solution, in which the components of the solution are shown in Table 5-1 to Table 5-3 (the sequences for primers, probes, and templates are replaced with the corresponding ones for L858R mutation), and the loading concentrations of the mutant templates are 0, 200, and 1,000 copies/μL respectively (the loading concentrations of the mutant templates can actually be adjusted according to the detection capability of the chip) and correspondingly, the loading concentrations of the wild-type templates are 30,000, 40,000, and 10,000 copies/μL respectively.

### 2. Chip Loading and Digital PCR Amplification

After the preparation of the solution, the chip was loaded and mounted. The loading volume was about 6.5-7 μL; and the temperature control program was set as follows: pre-denaturation at 95° C. for 5 mins; denaturation at 95° C. for 25 s, annealing at 55° C. for 35 s, extension at 72° C. for 60 s, for 42 cycles.

### 3. Fluorescent Signal Reading and Data Processing

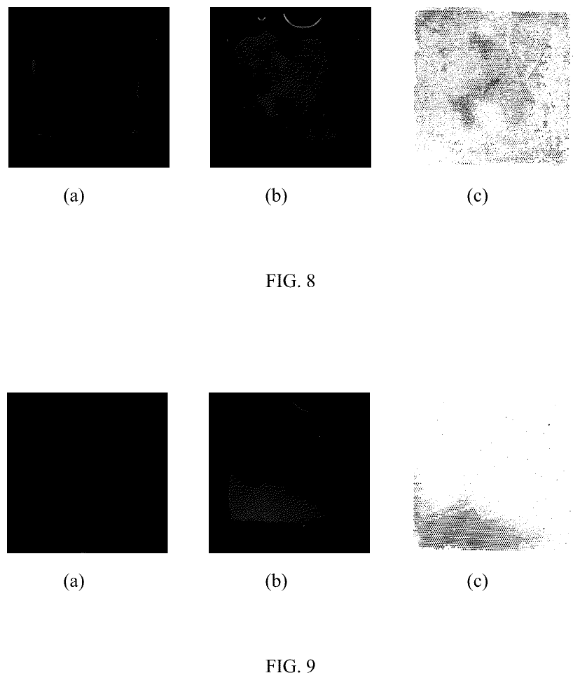

The chip was placed in the reader and fluorescent photos were taken, with the fluorescence channel set as FAM (excitation at about 488 nm, emission at about 525 nm), and the exposure time set as 2-5 s. The collected fluorescent dot matrix images were processed using ImageJ software, and the number of fluorescent bright spots (positive) was counted. Finally, the copy number concentration c of the sample was calculated according to the Poisson distribution formula c=−ln(1−p)/v, where p is the proportion of positive wells, and v is the volume of a single microwell. According to the results of the detection solution for the L858R 0.5% and 10% mutant systems, the measured concentrations of the mutant templates obtained from calculation are 387.7 copies/μL and 2,929 copies/μL, respectively, and the measured ratios of the mutant templates are 0.646% and 9.76%, respectively. Fluorescent signal results and data processing images are shown in FIG. 8 and FIG. 9.

The results indicate that it is effective to use the primers and probes for L858R mutation site of the present disclosure, and the reaction system can be used for the detection of samples containing this mutation site.

### Example 6. Detection of EGFR Gene Mutations in Paraffin-Embedded Tissue Samples from Lung Cancer Patients

The specific implementation steps are as follows:

1. DNA Extraction from Paraffin-Embedded Tissue Samples

DNA was extracted from paraffin-embedded tissue samples using TIANquick FFPE DNA kit from TIANGEN. The extraction process should be carried out in strict accordance with the instructions of the kit. The extracted nucleic acid samples can be used directly for subsequent experiments, or stored at −20° C. to avoid repeated freezing and thawing.

### 2. Amplification and Extraction of Target Fragments in EGFR Gene

(1) The target fragments in EGFR gene in the extracted nucleic acid samples were amplified using the primer pairs shown in Table 4. The amplification system is in 50 μL, including 10 μL of 5× Q5 polymerase buffer, 4 μL of 2.5 mM dNTPs, 2.5 μL of each of forward primer and reverse primer at 10 μM, 1 U of Q5 polymerase, 1-2 μL of DNA extraction solution from paraffin-embedded tissue, with the remaining of the volume supplemented with ddH₂O. The PCR program is set as follows: pre-denaturation at 98° C. for 2 mins; denaturation at 98° C. for 10 s, annealing at 62° C. for 20 s, extension at 72° C. for 20 s, for 30 cycles; incubation at 72° C. for 2 mins.

(2) After amplification, the amplified products were purified using the GeneJET PCR purification kit from Thermo Scientific. The purification process should be carried out in strict accordance with the instructions of the kit. The purified target nucleic acid fragments can be used directly for subsequent experiments, or stored at −20° C. to avoid repeated freezing and thawing.

### 3. Detection Solution Preparation

The components of the solution are as shown in Table 6.

TABLE 6
List of components of the detection solution for the purified
products extracted from the FFPE samples (clinical samples)

| | Stock Solution | | | | Final | |
|---|---|---|---|---|---|---|
| Component | Concentration | | Volume | | Concentration | |
| PCR master mix | 2X | | 6 | μL | 1.2X | |
| forward primer F | 9 | μM | 1 | μL | 900 | nM |
| reverse primer R | 9 | μM | 1 | μL | 900 | nM |
| Mutant Probe-MT-P | 5 | μM | 1 | μL | 500 | nM |

| | | | | |
|---|---|---|---|---|
| FFPE extraction and | 1.22 × 10⁵~2.50 × | 1 | μL | 1.22 × 10⁴~2.50 × |
| purification samples | 10⁵ c/μL | | | 10⁴ c/μL |
| total volume | / | 10 | μL | / |

### 4. Chip Loading and Digital PCR Amplification

After the preparation of the solution, the chip was loaded and mounted. The loading volume was about 6.5-7 μL; and the temperature control program was set as follows: pre-denaturation at 95° C. for 5 mins; denaturation at 95° C. for 25 s, annealing at 55° C. for 35 s, extension at 72° C. for 60 s, for 42 cycles.

### 5. Fluorescent Signal Reading and Data Processing

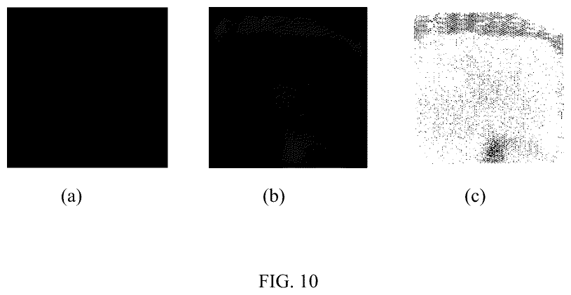

The chip was placed in the reader and fluorescent photos were taken, with the fluorescence channel set as FAM (excitation at about 488 nm, emission at about 525 nm), and the exposure time set as 2-5 s. The collected fluorescent dot matrix images were processed using ImageJ software, and the number of fluorescent bright spots (positive) was counted. Finally, the copy number concentration c of the sample was calculated according to the Poisson distribution formula c=−ln(1−p)/v, where p is the proportion of positive wells, and v is the volume of a single microwell. Fluorescent signal results and data processing images are shown in FIG. 10. The control method is the real-time fluorescent PCR (qPCR) method using the same fluorescent probes for qualitative determination. The calculated concentration of mutations in FFPE samples is 926.6 copies/μL, and the mutation ratio is 4.290%

1. A primer-probe composition for detecting an epidermal growth factor receptor (EGFR) gene mutation, comprising a combination of primers and probes for one or more of the following gene mutations: G719S point mutation at exon 18, E746_A750del deletion mutation at exon 19, T790M point mutation at exon 20, and L858R point mutation at exon 21.

2. The primer-probe composition of claim 1, wherein
for G719S point mutation at exon 18,
the primers comprise
SEQ ID NO: 3, SEQ ID NO: 4, or
a nucleotide sequence in which one or more nucleotides are substituted, deleted, added and inserted in the nucleotide sequence shown in SEQ ID NO: 3 or SEQ ID NO: 4 and
the probes comprise
SEQ ID NO: 5, SEQ ID NO: 6, or
a nucleotide sequence in which one or more nucleotides are substituted, deleted, added and inserted in the nucleotide sequence shown in SEQ ID NO: 5 or SEQ ID NO: 6,
wherein a first fluorescent group is conjugated to the 5′ end of the probe of SEQ ID NO: 5, and a minor groove binder is conjugated to the 3′ end of the probe of SEQ ID NO: 5,
wherein a second fluorescent group is conjugated to the 5′ end of the probe of SEQ ID NO: 6, and a minor groove binder is conjugated to the 3′ end of the probe of SEQ ID NO: 6.

3. A kit for detecting an EGFR gene mutation, comprising the primer-probe composition of claim 1, wherein the gene mutation is selected from one or more of G719S point mutation at exon 18, E746_A750del deletion mutation at exon 19, T790M point mutation at exon 20, and L858R point mutation at exon 21.

4. The kit of claim 3, further comprising a PCR master mix and a dNTP mix.

5. A method for detecting an EGFR gene mutation in a DNA sample based on digital PCR, wherein the gene mutation is selected from one or more of G719S point mutation at exon 18, E746_A750del deletion mutation at exon 19, T790M point mutation at exon 20, and L858R point mutation at exon 21, the method comprising:
i) providing the primer-probe composition of claim 1 for the EGFR gene mutation to be detected,
ii) mixing the DNA sample, the primer-probe composition provided in i), a PCR master mix and a dNTP mix to obtain a detection solution,
iii) loading the detection solution on a chip for PCR amplification reaction, and
iv) collecting and processing signals from the products of the PCR amplification reaction on the chip to detect whether the EGFR gene mutation is present in the DNA sample.

6. The method of claim 5, wherein the procedure of the PCR amplification reaction in iii) is
a) 92-98° C. for 2-8 minutes,
b) 92-98° C. for 15-40 seconds,
c) 52-58° C. for 20-50 seconds,
d) 68-76° C. for 40-80 seconds, and
e) 2-10° C. to terminate the reaction,
wherein the steps b)-d) are performed for 30-50 cycles.

7. The method of claim 5, wherein in the detection solution,
the DNA sample is at the final concentration of 300-30000 copies/μL,
both forward and reverse primers of the primers are at the final concentrations of 300-600 nM,
the probe is at the final concentration of 400-600 nM.

8. The method of any one of claim 7, wherein the mutation abundance in the DNA template is 0.5% or more.

9. A system for detecting an EGFR gene mutation, comprising the primer-probe composition of claim 1, wherein the gene mutation is selected from one or more of G719S point mutation at exon 18, E746_A750del deletion mutation at exon 19, T790M point mutation at exon 20, and L858R point mutation at exon 21.

10. A device for detecting an EGFR gene mutation in a DNA sample, comprising the system of claim 9.

11. Use of the primer-probe composition of claim 1 for detecting an EGFR gene mutation in a DNA sample, wherein the gene mutation is selected from one or more of G719S point mutation at exon 18, E746_A750del deletion mutation at exon 19, T790M point mutation at exon 20, and L858R point mutation at exon 21.

12. Use of the primer-probe composition of claim 1 for diagnosing diseases, wherein the diseases are cancers, for example lung cancer (such as lung adenocarcinoma, non-small cell lung cancer, or small cell lung cancer), hematologic malignancy, pancreatic cancer, colorectal cancer, or malignant glioma.

13. The primer-probe composition of claim 2, wherein
for E746_A750del deletion mutation at exon 19,
the primers comprise SEQ ID NO: 9, SEQ ID NO: 10, or
a nucleotide sequence in which one or more nucleotides are substituted, deleted, added and inserted in the nucleotide sequence shown in SEQ ID NO: 9 or SEQ ID NO: 10, and
the probes comprise SEQ ID NO: 11, SEQ ID NO: 12, or
a nucleotide sequence in which one or more nucleotides are substituted, deleted, added and inserted in the nucleotide sequence shown in SEQ ID NO: 11 or SEQ ID NO: 12,
wherein a first fluorescent group is conjugated to the 5′ end of the probe of SEQ ID NO: 11, and a minor groove binder is conjugated to the 3′ end of the probe of SEQ ID NO: 11, and
wherein a second fluorescent group is conjugated to the 5′ end of the probe of SEQ ID NO: 12, and a minor groove binder is conjugated to the 3′ end of the probe of SEQ ID NO: 12.

14. The primer-probe composition of claim 13, wherein
for T790M point mutation at exon 20,
the primers comprise SEQ ID NO: 15, SEQ ID NO: 16, or
a nucleotide sequence in which one or more nucleotides are substituted, deleted, added and inserted in the nucleotide sequence shown in SEQ ID NO: 15 or SEQ ID NO: 16, and
the probes comprise SEQ ID NO: 17, SEQ ID NO: 18, or
a nucleotide sequence in which one or more nucleotides are substituted, deleted, added and inserted in the nucleotide sequence shown in SEQ ID NO: 17 or SEQ ID NO: 18,
wherein a first fluorescent group is conjugated to the 5′ end of the probe of SEQ ID NO: 17, and a minor groove binder is conjugated to the 3′ end of the probe of SEQ ID NO: 17, and
wherein a second fluorescent group is conjugated to the 5′ end of the probe of SEQ ID NO: 18, and a minor groove binder is conjugated to the 3′ end of the probe of SEQ ID NO: 18.

15. The primer-probe composition of claim 14, wherein
for L858R point mutation at exon 21,
the primers comprise SEQ ID NO: 21, SEQ ID NO: 22, or
a nucleotide sequence in which one or more nucleotides are substituted, deleted, added and inserted in the nucleotide sequence shown in SEQ ID NO: 21 or SEQ ID NO: 22, and
the probes comprise SEQ ID NO: 23, SEQ ID NO: 24, or
a nucleotide sequence in which one or more nucleotides are substituted, deleted, added and inserted in the nucleotide sequence shown in SEQ ID NO: 23 or SEQ ID NO: 24,
wherein a first fluorescent group is conjugated to the 5′ end of the probe of SEQ ID NO: 23, and a minor groove binder is conjugated to the 3′ end of the probe of SEQ ID NO: 23, and
wherein a second fluorescent group is conjugated to the 5′ end of the probe of SEQ ID NO: 24, and a minor groove binder is conjugated to the 3′ end of the probe of SEQ ID NO: 24.

16. The primer-probe composition of claim 15, wherein the first fluorescent group and the second fluorescent group are respectively selected from Alexa Fluor 488, FAM, TET, JOE, VIC and HEX, and the first fluorescent group and the second fluorescent group are different.

17. Use of the kit of claim 3 for detecting an EGFR gene mutation in a DNA sample, wherein the gene mutation is selected from one or more of G719S point mutation at exon 18, E746_A750del deletion mutation at exon 19, T790M point mutation at exon 20, and L858R point mutation at exon 21.

18. Use of the system of claim 9 for detecting an EGFR gene mutation in a DNA sample, wherein the gene mutation is selected from one or more of G719S point mutation at exon 18, E746_A750del deletion mutation at exon 19, T790M point mutation at exon 20, and L858R point mutation at exon 21.

19. Use of the device of claim 10 for detecting an EGFR gene mutation in a DNA sample, wherein the gene mutation is selected from one or more of G719S point mutation at exon 18, E746_A750del deletion mutation at exon 19, T790M point mutation at exon 20, and L858R point mutation at exon 21.

20. Use of the kit of claim 3 for diagnosing diseases, wherein the diseases are cancers, for example lung cancer (such as lung adenocarcinoma, non-small cell lung cancer, or small cell lung cancer), hematologic malignancy, pancreatic cancer, colorectal cancer, or malignant glioma.
